SEQUENCE LISTING (2 sequences)

SEQ ID NO: 1 - DNA, 1437 bp, Artificial Sequence
  Synthetic (DNA sequence encoding CD63-GFP)
atggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatgacgcgtacgcggccgctcgagatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagagtt

SEQ ID NO: 2 - DNA, 5589 bp, Artificial Sequence
  Synthetic (pGL4.76_CMV_CD63-GFP sequence)
ggcctaactggccggtacctgagctcgctagcctcgaggatatcaagatctgccgccgcgatcgccatggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatgacgcgtacgcggccgctcgagatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagagttttctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctgcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgattcttctgacactagcgccaccatgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaaactctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

(19) United States
(12) Patent Application Publication
(10) Pub. No.: US 2013/0302856 A1
(43) Pub. Date: Nov. 14, 2013

(54) METHODS OF DIRECTLY EXTRACTING MICRORNA FROM MICROVESICLE IN CELL LINE, CELL CULTURE, OR BODY FLUID

(71) Applicant: SAMSUNG ELECTRONICS CO., LTD., Suwon-si (KR)

(72) Inventors: Chang-eun YOO, Seoul (KR); Ga-hee KIM, Yongin-si (KR); Myoung-soon KIM, Seoul (KR)

(73) Assignee: SAMSUNG ELECTRONICS CO., LTD., Suwon-si (KR)

(21) Appl. No.: 13/768,891

(22) Filed: Feb. 15, 2013

(30) Foreign Application Priority Data
May 9, 2012 (KR) 10-2012-0049279

(51) Int. Cl. C07H 1/08

(52) U.S. Cl. CPC C07H 1/08
USPC 435/91.2; 536/25.42

(57) ABSTRACT

A method of extracting a nucleic acid from a microvesicle, the method comprising treating the microvesicle with a composition comprising a detergent and an aprotic solvent to extract a nucleic acid from the microvesicle.

### CROSS-REFERENCE TO RELATED APPLICATIONS

This application claims the benefit of Korean Patent Application No. 10-2012-0049279, filed on May 9, 2012, in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 9,792 Byte ASCII (Text) file named “711801_ST25.txt,” created on Feb. 15, 2013.

### BACKGROUND

1. Field

The present disclosure relates to methods of extracting microRNA from microvesicles.

2. Description of the Related Art

In vivo microvesicles are small membranous vesicles that exist in various cell types or are secreted from cells. Microvesicles secreted into outside cells include: (i) exosomes, which are membranous vesicles having a diameter of 30 to 100 nm originated from phagocytic cells; (ii) ectosomes (also called shedding microvesicles (SMVs)), which are membranous vesicles that are directly separated from a plasma membrane and have a diameter of 50 to 1000 nm; and (iii) apoptotic blebs, which are vesicles secreted from dying cells and have a diameter of 50 to 5000 nm.

Among such microvesicles, under an electron microscope, it is confirmed that exosomes are not separated directly from a plasma membrane, but originate in a intracellular particular region called multivesicular bodies (MVBs) and are released and secreted to the outside cells. That is, once multivesicular bodies are fused with a plasma membrane, such vesicles are released to the outside cells. It is known that exosomes are separated and released from a plurality of other cell types under a normal state, a pathologic state, or a combination thereof. Although a molecular mechanism of such exosomes has not been revealed, it is known that, in addition to red blood cells, various kinds of immune cells, such as B-lymphocytes, T-lymphocytes, dendritic cells, blood platelets, and macrophages, as well as tumor cells produce and secret exosomes, when they are viable.

In vivo microvesicles (e.g., exosomes) include microRNA (miRNA), which is a useful marker in molecular diagnosis, such as early diagnosis of cancer. However, microvesicles are small in size and do not include a great amount of miRNA, while cells include a great amount of miRNA. Accordingly, a method of separating and purifying miRNA in microvesicles without loss is desired.

A typical method of separating miRNA from microvesicles includes, like those applied for most cells, lysis using a chaotropic salt, phenol-chloroform extraction, and silica extraction, which are sequentially performed in this stated order. This method may cause loss of miRNA due to the performance of several processes.

Accordingly, there is a need to develop a method of applying miRNA that is extracted by simply lysing only a membrane of a microvesicle to a subsequent process (for example, ligation or RT-qPCR) without a separate purification process.

### SUMMARY

Provided are methods of extracting a nucleic acid from a microvesicle in a sample, wherein the methods comprise, consist essentially of, or consist of separating a microvesicle from a sample; and treating the separated microvesicle with a composition, the composition comprising, consisting essentially of, or consisting of a detergent and an aprotic solvent.

Additionally, provided are methods of amplifying microRNA from a microvesicle in a sample, wherein the methods comprise, consist essentially of, or consist of separating a microvesicle from a sample; treating the separated microvesicle with a composition, wherein the composition comprises a detergent and an aprotic solvent; and amplifying the extracted nucleic acid by reverse transcription quantitative polymerase chain reaction (RT-qPCR). Preferably, RT-qPCR is performed without an additional purification process.

Related methods, compositions, and kits also are provided.

### BRIEF DESCRIPTION OF THE DRAWINGS

These and/or other aspects will become apparent and more readily appreciated from the following description of the embodiments, taken in conjunction with the accompanying drawings in which:

FIG. 1 illustrates a schematic diagram illustrating a process for extracting miRNA from a microvesicle (for example, exosomes) separated from a body fluid sample by using beads, wherein miRNA is separated by only lysis for direct use in a following process (a method as described herein), and a method using lysis, extraction, and purification (conventional method);

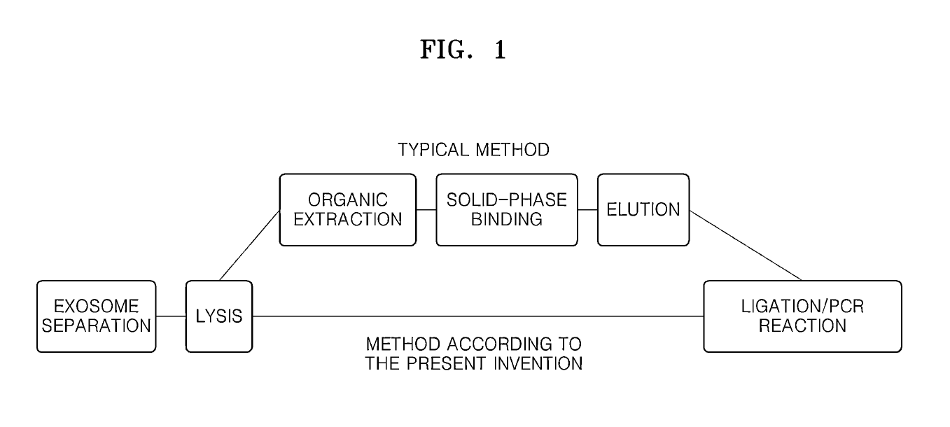

FIG. 2 shows scanning electron microscope (SEM) images of beads (a) before and (b) after a microvesicle is treated with a lysis solution;

FIG. 3 shows SEM images of microvesicles (a) before the treatment with a lysis solution; (b) after the treatment with a lysis solution manufactured by Invitrogen; (c) after the treatment with a TD lysis solution; and (d) after the treatment with a TF lysis solution.

FIG. 4 shows results of RT-qPCR on miRNA obtained from a microvesicle after lysis with TF lysis solution (TF), TD lysis solution, or the lysis solution (I) described in the Example 4. Crossing points (Cp) are indicated on the y-axis and Input EpCAM (ng) is indicated on the x-axis.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

The invention provides a composition for extracting a nucleic acid from a microvesicle, wherein the composition comprises, consists essentially of, or consists of a detergent and an aprotic solvent.

The term “detergent” refers to a material that is dissolved in a liquid to substantially decrease a surface tension. The detergent can be classified as an anionic detergent, a cationic detergent, a non-ionic detergent, and a zwitterionic detergent depending on the dissolved state in an aqueous solution. The non-ionic detergent may be, for example, TRITON™ X-100, polysorbate 20, polysorbate 40, polysorbate 60, polysorbate 80, or NP-40, but is not limited thereto.

The amount of the detergent in the composition is not particularly limited. Preferably, the composition contains about 0.1% (v/v) to about 5% (v/v) (e.g., about 0.5% (v/v), about 1% (v/v), about 1.5% (v/v), about 2% (v/v), about 2.5% (v/v), about 3% (v/v), about 3.5% (v/v), about 4% (v/v), or about 4.5% (v/v)) detergent.

The term “aprotic solvent” refers to a solvent that is not a protonic solvent. A protonic solvent refers to a solvent that dissociates in water, alcohols, carboxylic acids, or the like to produce protons and that forms a hydrogen bond between molecules. Examples of the aprotic solvent are acetone, acetonitrile, N,N-dimethylformamide (DMF), formamide, dimethyl sulfoxide (DMSO), and acetamide, but are not limited thereto.

The amount of the aprotic solvent in the composition is not particularly limited. Preferably, the composition contains about 1% (v/v) to about 20% (v/v) (e.g., about 2% (v/v), about 3% (v/v), about 4% (v/v), about 5% (v/v), about 6% (v/v), about 7% (v/v), about 8% (v/v), about 9% (v/v), about 10% (v/v), about 11% (v/v), about 12% (v/v), about 13% (v/v), about 14% (v/v), about 15% (v/v), about 16% (v/v), about 17% (v/v), about 18% (v/v), or about 19% (v/v) aprotic solvent.

The term “microvesicle” refers to a small membrane vesicle derived from cells. According to an embodiment of the invention, the microvesicle may be an exosome. According to another embodiment of the invention, the microvesicle may be derived from a cell line, a cell culture, or a body fluid.

The term “nucleic acid” refers to a macromolecule that consists of a purine base or a pyrimidine base, a sugar, and a phosphate. According to an embodiment of the invention, a nucleic acid contained in a microvesicle may be messenger RNA (mRNA) or microRNA (miRNA).

The invention provides a kit for extracting a nucleic acid from a microvesicle, wherein the kit comprises a composition comprising, consisting of, or consists of a detergent and an aprotic solvent.

The invention also provides a method of extracting a nucleic acid from a microvesicle in a sample, wherein the method comprises, consists essentially of, or consists of separating the microvesicle from the sample; and treating the separated microvesicle with a composition for the extraction of a nucleic acid, wherein the composition comprises a detergent and an aprotic solvent.

The method of extracting a nucleic acid from a microvesicle in a sample may be performed as follows:

First, the method may include separation of the microvesicle from the sample.

According to an embodiment of the invention, the sample may be any one selected from the group consisting of a cell line, a cell culture, blood, urine, mucus, saliva, tears, plasma, serum, sputum, spinal fluid, pleural effusion, nipple aspirate fluid, lymph, air duct fluid, intestinal juice, urogenital duct fluid, breast milk, lymphatic system fluid, semen, cerebrospinal fluid, bronchial fluid, ascites, cystic tumor fluid, and amniotic fluid, which are obtained from a body; and a combination thereof. However, the sample is not limited thereto as long as it includes microvesicles.

According to an embodiment of the invention, the separation of a microvesicle from a sample may be performed using a solid support or centrifugal force, a density gradient method, ultracentrifugation, filtering, dialysis, immunoaffinity column using an antibody, free flow electrophoresis, or a combination thereof. However, the separation method is not limited thereto, and any one of various methods for separating a microvesicle from a sample may be used. The solid support may include a material that binds specifically to a target material, and the target material may be EpCAM, CD63, CD81, or L1, but is not limited thereto. The material that binds specifically to a target material may be an antibody with respect to the target material, but is not limited thereto.

Thereafter, the separated microvesicle is treated with a composition for the extraction of a nucleic acid, wherein the composition comprises, consists essentially of, or consists of a detergent and an aprotic solvent. According to an embodiment of the invention, the treatment of the microvesicle with the composition for the extraction of a nucleic acid may be heating, but is not limited thereto. For example, the treatment may be performed by stirring, rotating, or vortexing while heating, and is not limited thereto.

The invention provides a method of extracting a nucleic acid from a microvesicle in a sample, wherein the method comprises, consists essentially of, or consists of separating the microvesicle from the sample; treating the separated microvesicle with a composition for the extraction of a nucleic acid, wherein the composition comprises, consists essentially of, or consists of a detergent and an aprotic solvent; and amplifying the extracted nucleic acid by reverse-transcription quantitative polymerase chain reaction (RT-qPCR) (preferably without purification).

The composition for extraction of a nucleic acid “consists essentially of” a detergent, aprotic solvent, and other stated components if it does not contain other components that would materially interfere with the amplification of the nucleic acid in process like polymerase chain reaction (RT-qPCR) when no further purification of the nucleic acid is performed between extraction and amplification.

The method of amplifying a microRNA from a microvesicle in a sample may be performed as described below.

First, the method may include the separation of the microvesicle from the sample.

Thereafter, the separated microvesicle is treated with a composition for the extraction of a nucleic acid, wherein the composition comprises, consists essentially of, or consists of a detergent and an aprotic solvent.

Thereafter, the extracted nucleic acid is amplified by RT-qPCR.

The term “RT-qPCR (reverse transcription quantitative polymerase chain reaction)” refers to real-time PCR amplification of RNA into complementary DNA (cDNA) that is complementary to the RNA by using a reverse transcriptase. According to an embodiment of the invention, miRNA obtained from a microvesicle may be used for RT-qPCR without an additional purification process such as an extraction process with an organic solvent or an extraction process including binding to a solid support. The organic solvent may be phenol, chloroform, or a mixture thereof, but is not limited thereto. The solid support may be silica, but is not limited thereto.

Hereinafter, embodiments of the invention are described in detail with reference to examples. However, the examples are presented herein for illustrative purpose only, and do not limit the scope of the invention.

### EXAMPLE 1

### Preparation of Beads for Separation of miRNA from Sample

### 1-1. Coupling of Polymer with Carboxylic Acid on Surface of Magnetic Beads

100 μL of Dynabeads M-270 Amine (Invitrogen) were washed twice with 200 μL of a buffer solution (0.1 M 2-morpholinoethanesulfonic acid (MES), 0.5 M NaCl, pH 6.0), and then, re-suspended with 100 μL of a buffer solution. 48 μL of a 35% w/v polyacrylic acid (Aldrich) solution diluted by 1/10 and 236 μL of a buffer solution were mixed and then, the mixture was added to the beads, followed by homogeneously mixing.

Thereafter, 54 μL of a 75 mg/mL ethyl-3-dimethyl-aminopropyl carbodiimide (EDC) solution (in distilled water), and 210 μL of a 15 mg/mL N-hydroxysuccinimide (NHS) solution (in distilled water) were added thereto and the result was rotated for one hour. Then, washing was performed thereon twice with 400 μL of a buffer solution and then the result was re-suspended with 400 μL of a buffer solution.

### 1-2. Coupling of Protein G on Surface of Magnetic Beads and Surface Treatment

The bead solution prepared according to Example 1-1 was washed twice with 400 μL of buffer solution (0.025 M MES, pH 6.0). 54 μL of a 75 mg/mL EDC solution (in 0.025 M MES, pH 6.0), 210 μL of a 15 mg/mL NHS solution (in 0.025 M MES, pH 6.0), and 236 μL of a buffer solution were added to the beads and then mixed well, followed by 30 minutes of rotation. The beads were washed twice with 400 μL of a buffer solution and then re-suspended with 400 μL of a buffer solution. Then, 3 μL of a protein G solution (10 μg/μL) was added thereto, and the mixture was rotated for one hour. Thereafter, 100 μL of sulfobetaine (SB, 100 μg/μL in distilled water) was added thereto and the mixture was rotated for 1 to 2 hours. Then, the result was washed twice with 400 μL of 1×PBS (0.02% tween) and twice with 400 μL of 1×PBS.

### 1-3. Conjugation of Protein G and Antibody

The bead solution prepared according to Example 1-2 was washed twice with 400 μL of a buffer solution (0.1 M sodium acetate, pH 5.0). 160 μL of anti-EpCAM (R&D systems, 0.5 μg/μL in 1×PBS) and 340 μL of a buffer solution were mixed. Then, the mixed solution was added to the beads, followed by three hours of rotation. Thereafter, the result was washed twice with 200 μL of 1×PBS (0.02% tween) and washed twice with 200 μL of 1×PBS, followed by re-suspension of 100 μL of 1×PBS.

### 1-4. Crosslinking Between Protein G and Antibody

The bead solution prepared according to Example 1-3 was washed twice with 400 μL of a buffer solution (0.1 M sodium borate, pH 9.3). 400 μL of 20 mM DMP (in a buffer solution, pH 9.3) was added to beads, followed by one hour of rotation. Thereafter, the result was washed twice with 400 μL of a buffer solution (50 mM ethanolamine, 0.1 M of sodium borate, pH 8.0), and then 200 μL of a buffer solution was added thereto and the result was rotated for one hour. Thereafter, the result was washed twice with 200 μL of 1×PBS (0.02% tween) and washed twice with 200 μL of 1×PBS, followed by re-suspension of 100 μL of 1×PBS.

### EXAMPLE 2

### Separation of Microvesicle (Exosome) in Cell Culture Medium

All of the following processes were performed in ice or at 4° C.

A cell culture medium was placed in a 50 mL centrifugal tube. The cell culture was centrifuged at 300×g at 4° C. for 10 minutes. A supernatant was separated and placed in a new centrifugal tube, and the supernatant was centrifuged at 800×g at 4° C. for 10 minutes. The supernatant was separated and placed in a new centrifugal tube, and the supernatant was centrifuged at 2000×g at 4° C. for 20 minutes. The supernatant was separated and placed in a polycarbonate tube, and the supernatant was centrifuged at 10,000×g at 4° C. for 30 minutes. The supernatant was separated and placed in a polycarbonate tube, and the supernatant was centrifuged at 110,000×g at 4° C. for 70 minutes. The supernatant was completely removed and the result was re-suspended with 1 mL PBS. The suspension was centrifuged at 100,000×g at 4° C. for 70 minutes. The supernatant was completely removed. An amount of total protein and EpCAM was quantified by bicinchoninic acid (BCA) method (Pierce) and Western blotting and stored at −70° C. before use.

### EXAMPLE 3

### Preparation of Lysis Solution

1.61 g NaCl was dissolved in 50 mL of a 1×PBS solution, and then 2.5 mL of a 10% TRITON™ X-100 solution was added thereto. 1 mL of DMSO or 1 mL of formamide was added to 9 mL of the result solution (TRITON™ X-100 and DMSO will be indicated as ‘TD’, and TRITON™ X-100 and formamide will be indicated as ‘TF’).

The lysis solution was prepared in consideration of a lysis efficiency of microvesicle, inhibition on miRNA, and conditions under which miRNA is not adsorbed to beads.

### EXAMPLE 4

### Measuring of Lysis Efficiency of Microvesicle (Exosome) According to Lysis Solution

### 4-1. Preparation of GFP-Labeled Microvesicle (Exosome)

For preparation of an microvesicle (exosome) including CD63-GFP fusion protein, a vector (SEQ ID NO: 2; pGL4.76_CMV_CD63-GFP nucleotide sequence) encoding a fusion protein of CD63 and GFP (Green fluorescence protein) was manufactured by inserting a CMV promoter and nucleotides encoding CD63-GFP fusion protein (SEQ ID NO: 1; CD63-GFP nucleotide sequence) at a multicloning site (MSC) in pGL4.76(AY864931) plasmid as a template.

One day before transfection, cells were uniformly inoculated and cultured on a 150 mm plate. 7.5 μg of plasmid DNA was diluted in 7.5 ml of an Opti-MEM serum-free medium (Invitrogen) and then, completely mixed. Plus reagent (Invitrogen) was completely mixed before use, and then, 75 μL of the plus reagent was added to the diluted DNA, and then slowly mixed and incubated at room temperature for 5 minutes. Lipofectamine™ LTX was smoothly mixed before use, and then, 187.5 μL thereof was directly added to the incubated mixed solution and then completely mixed. Thereafter, the cells were incubated at room temperature for 30 minutes.

The DNA-lipid composite was added dropwise to the plate with MCF-7 cells (ATCC) that were to be transfected. Then, mixing was performed thereon while slowly shaking the plate. The plate, on which the DNA-lipid composite was mixed with cells, was incubated at 37° C., in a CO₂ incubator for 12 to 24 hours. Thereafter, the cells were placed in an exosome-free medium. A culture medium with fetal bovine serum (FBS) was exchanged with a medium containing an exosome-free FBS. The cells were cultured in a CO₂ incubator at 37° C. for 24 to 48 hours, and then, the conditioned medium was collected.

A clean conditioned medium was placed in a 50 μL centrifugal tube, and then, centrifuging was performed thereon at 4° C. and at 300×g for 10 minutes. After the supernatant was removed, the residue was placed in a new centrifugal tube. Centrifuging was performed thereon at 4° C. and at 300×g for 10 minutes. After the supernatant was removed, the residue was placed in a new centrifugal tube. Centrifuging was performed thereon at 4° C. and at 2,000×g for 20 minutes. The supernatant was placed in a polyallomer tube or a polycarbonate vial, which is suitable for a super-speed centrifugal separator. Centrifuging was performed thereon at 4° C. and at 10,000×g for 30 minutes. The supernatant was placed in a tube suitable for a super-speed centrifugal separator. The supernatant was centrifuged at 4° C. and at 110,000×g for 70 minutes, and then, the supernatant was completely removed. Pellets were re-suspended with 1000 μL PBS in a tube. Then, the tube was filled with PBS, followed by centrifuging at 4° C. and at 100,000×g for 70 minutes. The supernatant was removed as completely as possible. Pellets were re-suspended with PBS in a tube, followed by centrifuging at 4° C. and at 100,000×g for 70 minutes. The supernatant was removed as completely as possible. To re-suspend pellets, a small amount of PBS or TBS was added thereto and then re-suspension was performed thereon. The result was fractioned in an amount of 100 μL, and preserved at −80° C., and when needed, melted for use.

### 4-2. Separation of Microvesicle (Exosome) in Serum

300 μL of a solution in which human serum (Sigma) was mixed with prepared GFP-labeled exosome was added to 30 μL of prepared beads, and then the mixture was rotated for 3 hours at a rotational rate of 30 rpm. The supernatant was removed, the residue was washed three times with 200 μL of 1×PBS, and then rotated for 3 hours in 300 μL of 1×PBS. The result supernatant was removed, the residue was washed three times with 200 μL of 1×PBS, and then beads were separated by using a magnet.

### 4-3. Lysis of Separated Microvesicle (Exosome)

20 μL of a TD or a TF solution was added to the prepared beads to which GFP-labeled microvesicle (exosome) bound, and then subjected to vortexing every 10 minutes while heating at 60° C. for 40 minutes. The result was centrifuged for 5 seconds at a rotational rate of 1000 rpm, and then beads and a solution were separated from each other using a magnet. In addition, as a comparative experiment, 300 μL of a lysis solution contained in a PureLink miRNA separation kit manufactured by Invitrogen was added to the same beads, vortexing was performed thereon for 1 minute, and then the lysis solution and beads were separated by using a magnet.

### 4-4. Lysis Efficiency Measurement

100 μL of a GFP assay buffer solution (BioVision) was added to the separated beads and beads that were not subjected to the lysis treatment. Then, the respective bead solutions separately were mixed well. A reaction was allowed to progress for 10 minutes at room temperature, and then the lysis solution and the beads were separated. The fluorescence intensity of each solution was measured using Beckman Couler DTX 800. Fluorescent intensity values before and after the treatment with the lysis solution were compared to calculate a lysis efficiency.

Table 1 includes the lysis efficiencies when GFP-labeled microvesicles (exosomes) separated using beads were treated with lysis solutions.

TABLE 1

| Lysis solution | Invitogen | TD | TF |
|---|---|---|---|
| Lysis efficiency (%) | 99.6 | 97.7 | 99.3 |

It was confirmed that the lysis solution prepared in Example 3 enables lysis of microvesicles (exosomes) binding to beads at an equivalent level as a commercially available lysis solution using a chaotropic salt.

### EXAMPLE 5

### Measuring of Scanning Electron Microscope (SEM) Images of Microvesicles (Exosomes)

### 5-1. Separation of Microvesicles (Exosomes) in Serum

300 μL of a solution, in which human serum (Sigma) was mixed with the microvesicle (exosome) prepared according to Example 2, was added to 30 μL of beads prepared according to Example 1. Then, the mixture was rotated for 24 hours at a rotational rate of 30 rpm. The supernatant was removed, and the residue was washed three times with 200 μL of 1×PBS and then rotated for 3 hours in 300 μL of 1×PBS. The result supernatant was removed, the residue was washed three times with 200 μL of 1×PBS, and then beads were separated by using a magnet.

### 5-2. Lysis of Separated Microvesicles (Exosomes)

20 μL of a TD or a TF solution was added to the beads to which microvesicles (exosomes) bound, prepared according to Example 5-1. The resulting solution was subjected to vortexing every 10 minutes while heating at 60° C. for 40 minutes. The result was centrifuged for 5 seconds at 1000 rpm, and then beads and a solution were separated by using a magnet. In addition, as a comparative experiment, 300 μL of a lysis solution contained in a PureLink miRNA separation kit manufactured by Invitrogen was added to the same beads, vortexing was performed for 1 minute, and then the lysis solution and beads were separated from each other by using a magnet.

### 5-3. SEM Image Measuring

SEM images of the separated beads and solution were measured. A copper grid was placed on a 0.22 μm filter and then 10 μL of the sample manufactured according to Example 5-2 was dropped thereon. The result was washed three times with deionized (DI) water, and then 4% glutaraldehyde was dropped thereon, and dried for 30 minutes at room temperature, thereby fixed. Thereafter, washing was performed three times thereon with DI water, and then dehydrated with 70% ethyl alcohol. Thereafter, the result was dehydrated with 100% ethyl alcohol and then dried in an oven at 37° C. for 2 hours or more. The prepared sample was fixed on a carbon tape and vacuum-coated with OSO₄ for 30 minutes. The surface of the sample was confirmed by using SEM (S-5500, Hitachi, Tokyo, Japan).

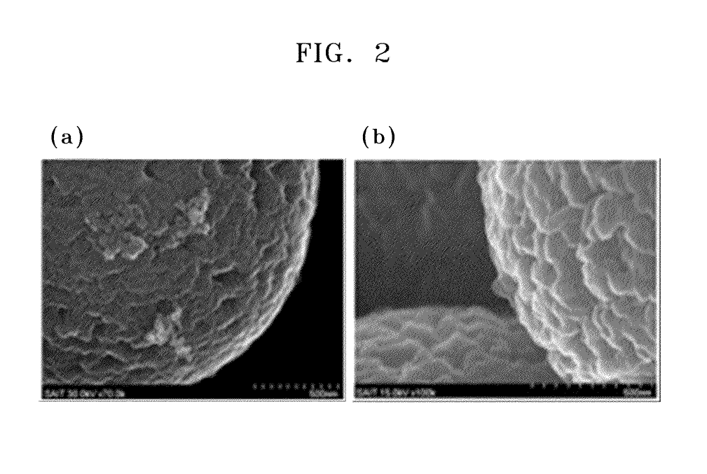

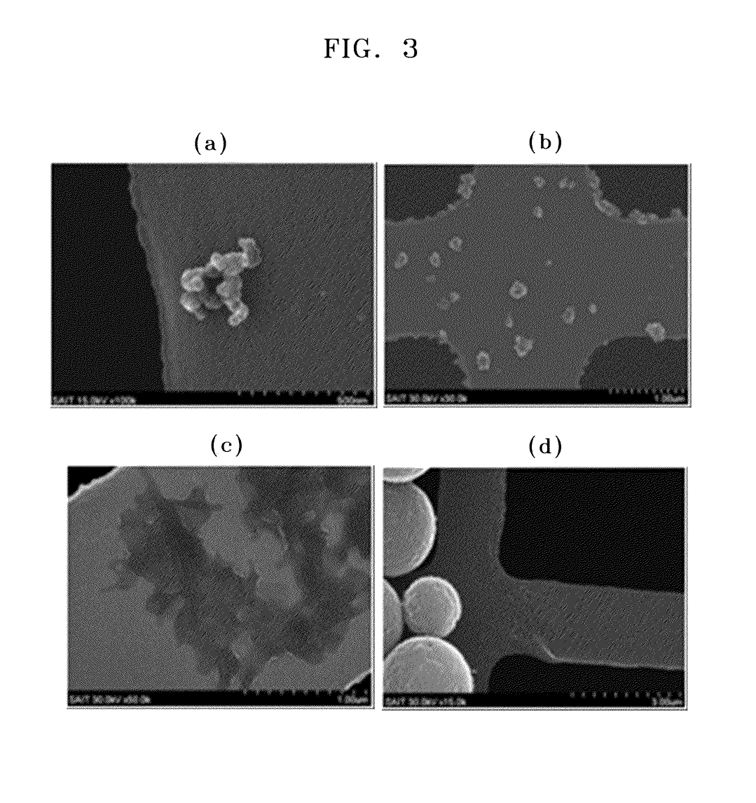

Microvesicles (exosomes) were separated by using beads and then treated with lysis solutions. FIGS. 2 and 3 show SEM images of beads and lysis solutions, respectively.

From the SEM images of the surface of beads, it was confirmed that the microvesicles (exosomes) binding to the beads were separated from the beads after the treatment with lysis solutions (FIG. 3). This was true for all of the lysis solutions.

From the SEM images of the solutions, it was confirmed that after the lysis, materials, such as microvesicles (exosomes), which were identified before the treatment with the lysis solutions were no longer present, and various types of aggregation were observed based on the solution used. In particular, when treated with a lysis solution, a microvesicle was not separated from beads, but lysed to form an aggregation of protein and membrane residue.

### EXAMPLE 6

### RT-qPCR of Microvesicle (Exosome)-Lysed Mixture

### 6-1. Separation of Microvesicle (Exosome) in Serum and Lysis of Separated Microvesicle

Microvesicles (exosomes) in serum were separated by using beads in the same manner as in Example 5-1. Then, the microvesicles were dissolved in the same manner as in Example 5-2.

### 6-2. RT-qPCR

The mixture obtained by the treatment with TD or TF in Example 6-1 was directly used for RT-qPCR without a separate purification process. For comparison purposes, a mixture treated with a separation kit manufactured by Invitrogen was used for RT-qPCR after a purification process was performed according to the instruction in the manual.

RT-qPCR was performed by using a Taqman miRNA assay kit manufactured by Applied Biosystems according to the instructions in the manual. Reverse transcription was performed by using Tetrad® 2 (BioRad), and qPCR was performed by using Roche LightCycler® LC-480.

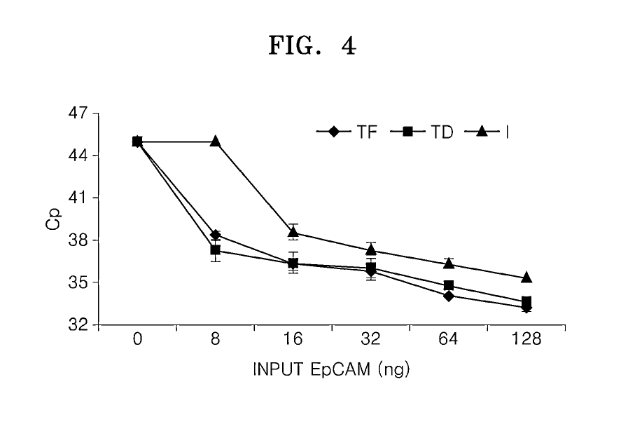

Microvesicles (exosomes) in serum were captured by using beads at various amounts of input EpCAM. miRNA was obtained by using various lysis methods. RT-qPCR was performed with respect to miR-200c, and crossing points (Cp) values were calculated. The results are shown in FIG. 4 and Table 2.

TABLE 2

| | Solution | | |
|---|---|---|---|
| Inputd EpCAM (ng) | Invitogen | TD | TF |
| 0 | 45* | 45 | 45 |
| 8 | 45 | 37.4(±0.8) | 38.4(±0.3) |
| 16 | 38.6(±0.5) | 36.4(±0.4) | 36.5(±0.7) |
| 32 | 37.5(±0.4) | 36.1(±0.7) | 35.8(±0.6) |
| 64 | 36.5(±0.3) | 34.9(±0.4) | 34.1(±0.1) |
| 128 | 35.4(±0.0) | 33.7(±0.0) | 33.3(±0.3) |

*Cp values were not actually measured; rather, they were indicated as a maximum cycle number when qPCR was performed.

In all lysis conditions, whenever an amount of the introduced EpCAM doubled, Cp values decreased by about 1. This means that under all lysis conditions, extraction and detection were quantified. In addition, compared to the lysis method presented by Invitrogen (conventional method), when TD and TF were used for the lysis according to the inventive methods, the Cp value was as small as about 2. Even when the amount of the introduced EpCAM was 8 ng, miRNA was quantitatively detected.

Accordingly, a method according to the invention enables quantitative extraction of miRNA from a microvesicle (exosome) separated from a body fluid sample, and a detection limit is higher than when a commercially available kit is used.

A composition for the extraction of a nucleic acid from a microvesicle (exosome), wherein the composition comprises a detergent and an aprotic solvent, according to embodiments of the invention, enables one-step extraction of miRNA from a microvesicle (exosome) from a cell line, a cell culture, or a body fluid sample, thereby allowing use of the extracted miRNA in a subsequent process, such as ligation or RT-qPCR, without further purification steps. In addition, detection sensitivity of miRNA may be improved by adjusting a composition of a lysis solution of microvesicles. The inventive compositions and kits may also be used in analyzing, in addition to the microvesicle, a particle having a lipid double layer, for example, a nucleic acid in cells.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of extracting a nucleic acid from a microvesicle in a sample, the method comprising:
separating a microvesicle from a sample; and
treating the separated microvesicle with a composition comprising a detergent and an aprotic solvent to extract a nucleic acid from the microvesicle.

2. The method of claim 1, wherein the sample is a cell line, a cell culture, or a body fluid.

3. The method of claim 2, wherein the body fluid is serum.

4. The method of claim 1, wherein the separating is performed by using a solid support or a centrifugal force.

5. The method of claim 4, wherein the solid support comprises a material that binds specifically to a target material of a microvesicle.

6. The method of claim 1, wherein the detergent is TRITON™ X-100.

7. The method of claim 1, wherein the aprotic solvent is selected from the group consisting of formamide, dimethyl sulfoxide (DMSO), and acetamide.

8. The method of claim 1, wherein the treating is heating.

9. The method of claim 1, wherein the microvesicle is an exosome.

10. The method of claim 1, wherein the nucleic acid is a microRNA (miRNA).

11. A method of amplifying a microRNA from a microvesicle in a sample, the method comprising:
extracting a nucleic acid from a microvesicle according to claim 1; and
amplifying the extracted nucleic acid by reverse transcription quantitative polymerase chain reaction (RT-qPCR).

12. The method of claim 11, wherein RT-qPCR is performed without an additional purification process.

13. The method of claim 11, wherein the detergent is TRITON™ X-100.

14. The method of claim 11, wherein the aprotic solvent is selected from the group consisting of formamide, dimethyl sulfoxide (DMSO), and acetamide.

15. The method of claim 11, wherein the nucleic acid extracted by contact with a composition comprising a detergent and an aprotic solvent is amplified without further purification.
